(19) United States
(12) Patent Application Publication
(10) Pub. No.: US 2022/0054572 A1
(43) Pub. Date: Feb. 24, 2022

(54) PHARMACEUTICAL COMPOSITIONS AND USE THEREOF

(71) Applicant: SANDALWOOD BIOTECH INC., Fremont, CA (US)

(72) Inventors: Be XIE, Fremont, CA (US); Xuefeng REN, Buffalo, NY (US)

(21) Appl. No.: 17/409,301

(22) Filed: Aug. 23, 2021

Related U.S. Application Data
(60) Provisional application No. 63/069,511, filed on Aug. 24, 2020.

(51) Int. Cl. A61K 36/736; A61K 36/17; A61K 33/06; A61K 36/484; A61K 45/06; A61K 9/00; A61P 31/16

(52) U.S. Cl. CPC A61K 36/736; A61K 36/17; A61K 33/06; A61K 36/484; A61K 2236/331; A61K 9/0053; A61P 31/16; A61K 2236/17; A61K 45/06

(57) ABSTRACT

The present application relates to methods of preventing, treating or managing a respiratory disease and/or a viral infection (e.g., an influenza virus infection and COVID-19) using a pharmaceutical composition produced from one or more preparations of plant parts. Provided herein are the pharmaceutical compositions produced from the one or more preparations. Also provided herein are methods of producing the pharmaceutical composition from the one or more preparations.

This application claims priority to U.S. Provisional Application No. 63/069,511, filed on Aug. 24, 2020, the entirety of which is incorporated herein by reference.

### 1. INTRODUCTION

The present application relates to methods of preventing, treating or managing a respiratory disease and/or a viral infection (e.g., an influenza virus infection and COVID-19) using a pharmaceutical composition produced from one or more preparations of plant parts. Provided herein are the pharmaceutical compositions produced from the one or more preparations. Also provided herein are methods of producing the pharmaceutical compositions.

### 2. BACKGROUND

Seasonal influenza is an acute respiratory infection caused by influenza viruses which circulate in all parts of the world. There are 4 types of seasonal influenza viruses, types A, B, C and D. Influenza A and B viruses circulate and cause seasonal epidemics of disease. Influenza can cause severe illness or death especially in people at high risk. Up to 650,000 deaths annually are associated with respiratory diseases from seasonal influenza, according to estimates by the United States Centers for Disease Control and Prevention (US-CDC), WHO and global health partners in 2017. In industrialized countries, epidemics can result in high levels of worker/school absenteeism and productivity losses. Clinics and hospitals can be overwhelmed during peak illness periods. Research estimates that 99% of deaths in children under 5 years of age with influenza related lower respiratory tract infections are found in developing countries (Nair H, et al. Lancet 2011; 378: 1917-3).

Coronavirus disease 2019 (COVID-19) is an infection caused by severe acute respiratory syndrome coronavirus 2 (SARS-CoV-2). The novel coronavirus outbreak of SARS-CoV-2, as of May 14, 2020, has resulted in more than 4,300,000 individuals infected and over 290,000 deaths worldwide (Dong E, et al., Lancet Infect. Dis. 2020; 20: 533-34). The current pandemic has brought to the forefront urgency and necessity for rapid development of pandemic countermeasures (Sempowski G D, et al. Cell 2020; S0092-8674 (20) 30674-7).

### 3. SUMMARY OF THE INVENTION

Provided herein are methods of preventing, treating or managing a respiratory disease and/or a viral infection (e.g., an influenza virus infection and COVID-19) using a pharmaceutical composition produced from one or more preparations of plant parts. Also provided herein are pharmaceutical compositions produced from one or more preparations. Also provided herein are methods of producing pharmaceutical compositions.

In one aspect, provided herein are methods of preventing, treating or managing a respiratory disease and/or a viral infection (e.g., an influenza virus infection and COVID-19). In certain embodiments, provided herein is a method of preventing, treating or managing a respiratory disease and/or a viral infection, comprising administering to a subject in need of such prevention, treatment, or management a pharmaceutical composition produced from one or more preparations comprising

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the respiratory disease is pneumonia.

In certain embodiments, the respiratory disease and/or the viral infection is an influenza virus infection. In certain embodiments, the influenza virus is influenza virus A.

In certain embodiments, the respiratory disease and/or the viral infection is a coronavirus infection. In certain embodiments, the coronavirus is the severe acute respiratory syndrome coronavirus 2 (SARS-CoV-2). In certain embodiments, the coronavirus infection is coronavirus disease 2019 (COVID-19).

In certain embodiments, the subject has symptoms comprising fever (body temperature at about 99.5° F. or above), dry cough, and/or difficulty breathing. In certain embodiments, the subject shows abnormality in a chest computed tomography (CT) scan.

In certain embodiments, the method treats and/or manages the respiratory disease and/or the viral infection.

In certain embodiments, provided herein is a method of preventing, treating or managing COVID-19, comprising administering to a subject in need of such prevention, treatment, or management a pharmaceutical composition produced from one or more preparations comprising:

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the method treats and/or manages COVID-19.

In certain embodiments, the method further comprises administering to the subject a second agent. In certain embodiments, the second agent is an antiviral agent, an anti-inflammatory agent, a Janus kinase inhibitor, a steroid hormone, an immunosuppressive agent, an agent targeting interleukin-6 receptor, an antibacterial agent, an anti-parasitic agent, Traditional Chinese medicine, stem cells, convalescent plasma, or a combination thereof. In certain embodiments, the second agent is remdesivir, LPV/r (lopinivir-ritonavir), arbidol, favipiravir, baricitinib, corticosteroids, interferon, tocilizumab, sarilumab, azithromycin, hydroxychloroquine or chloroquine, or a combination thereof.

In certain embodiments, the pharmaceutical composition is administered via oral route.

In certain embodiments, the preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %; and/or
(iv) the preparation of the dried root and rhizome of Glycyrrhiza uralensis Fisch. in an amount of 1 wt. % to 50 wt. %.

In certain embodiments, the preparations comprise:

(i) the preparation of the grassy stem of Ephedra sinica Stapf in an amount of 2 g to 20 g;
(ii) Gypsum ore (CaSO₄.2H₂O) in an amount of 15 g to 60 g;
(iii) the preparation of the seed (nut) of Semen Armeniacae Amarum in an amount of 5 g to 20 g; and/or
(iv) the preparation of the dried root and rhizome of Glycyrrhiza uralensis Fisch. in an amount of 3 g to 20 g.

In certain embodiments, the preparations comprise:

(i) the preparation of the grassy stem of Ephedra sinica Stapf in an amount of 10 g;
(ii) Gypsum ore (CaSO₄.2H₂O) in an amount of 30 g;
(iii) the preparation of the seed (nut) of Semen Armeniacae Amarum in an amount of 10 g; and/or
(iv) the preparation of the dried root and rhizome of Glycyrrhiza uralensis Fisch. in an amount of 10 g.

In certain embodiments, the preparations further comprise:

(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

In certain embodiments, the preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %.

In certain embodiments, the preparations comprise

(v) the preparation of the seed kernel of Coix lacryma-jobi L. var. mayuen (Roman.) Stapf in an amount of 9 g to 30 g;
(vi) the preparation of the dried sclerotia of Poria cocos (Schw.) Wolf in an amount of 5 g to 35 g and
(vii) the preparation of the dried root of Pueraria lobata Ohwi in an amount of 5 g to 35 g.

In certain embodiments, the preparations comprise:

(v) the preparation of the seed kernel of Coix lacryma-jobi L. var. mayuen (Roman.) Stapf in an amount of 15 g;
(vi) the preparation of the dried sclerotia of Poria cocos (Schw.) Wolf in an amount of 20 g and
(vii) the preparation of the dried root of Pueraria lobata Ohwi in an amount of 20 g.

In another aspect, provided herein are pharmaceutical compositions produced from one or more preparations.

In certain embodiments, provided herein is a pharmaceutical composition produced from preparations comprising:

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %; and/or
(iv) the preparation of the dried root and rhizome of Glycyrrhiza uralensis Fisch. in an amount of 1 wt. % to 50 wt. %.

In certain embodiments, the preparations comprise:

(i) the preparation of the grassy stem of Ephedra sinica Stapf in an amount of 2 g to 20 g;
(ii) Gypsum ore (CaSO₄.2H₂O) in an amount of 15 g to 60 g;
(iii) the preparation of the seed (nut) of Semen Armeniacae Amarum in an amount of 5 g to 20 g; and/or
(iv) the preparation of the dried root and rhizome of Glycyrrhiza uralensis Fisch. in an amount of 3 g to 20 g.

In certain embodiments, the preparations comprise:

(i) the preparation of the grassy stem of Ephedra sinica Stapf in an amount of 10 g;
(ii) Gypsum ore (CaSO₄.2H₂O) in an amount of 30 g;
(iii) the preparation of the seed (nut) of Semen Armeniacae Amarum in an amount of 10 g; and/or
(iv) the preparation of the dried root and rhizome of Glycyrrhiza uralensis Fisch. in an amount of 10 g.

In certain embodiments, the preparations comprise:

(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

In certain embodiments, the preparations comprise

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %.

In certain embodiments, the preparations comprise

(v) the preparation of the seed kernel of Coix lacryma-jobi L. var. mayuen (Roman.) Stapf in an amount of 9 g to 30 g;
(vi) the preparation of the dried sclerotia of Poria cocos (Schw.) Wolf in an amount of 5 g to 35 g and
(vii) the preparation of the dried root of Pueraria lobata Ohwi in an amount of 5 g to 35 g.

In certain embodiments, the preparations comprise:

(v) the preparation of the seed kernel of Coix lacryma-jobi L. var. mayuen (Roman.) Stapf in an amount of 15 g;
(vi) the preparation of the dried sclerotia of Poria cocos (Schw.) Wolf in an amount of 20 g and
(vii) the preparation of the dried root of Pueraria lobata Ohwi in an amount of 20 g.

In another aspect, provided herein are methods of producing pharmaceutical compositions.

In certain embodiments, provided herein is a method of producing a pharmaceutical composition, wherein the method comprises the steps of:

(a) boiling preparations in water; and
(b) collecting the decoction,
wherein the preparations comprise:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the method further comprises, before step (a), a step of soaking preparation in water for a period of time.

In certain embodiments, the method further comprises, after step (b), a step of mixing water with the remaining preparations, boiling the mixture of water and the remaining preparations, collecting the second decoction, and mixing the second decoction with the decoction collected in step (b).

In certain embodiments, provided herein is a method of producing a pharmaceutical composition, wherein the method comprises the steps of:

(a) boiling preparations in water;
(b) collecting the decoction;
(c) filtering the decoction and collecting the filtrate; and
(d) freeze-drying the filtrate to obtain a dry powder;
wherein the preparations comprise:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the method further comprises, after step (b) and before step (c), a step of boiling the remaining preparations in water again, collecting the second decoction, and mixing the second decoction with the decoction collected in step (b) to produce the decoction in step (c).

In certain embodiments, after each step of boiling and before each step of collecting the decoction, the preparations in water are maintained at a temperature of about 60° C. to about 100° C. for a period of about 30 minutes to about 120 minutes. In certain embodiments, the method further comprises, after step (c) and before step (d), a step of concentrating the filtrate under reduced pressure. In certain embodiments, the method further comprises, after step (d), a step of mixing the dry powder with an encapsulating agent to produce a granule. In certain embodiments, the encapsulating agent is beta-cyclodextrin.

In certain embodiments, the preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %; and/or
(iv) the preparation of the dried root and rhizome of Glycyrrhiza uralensis Fisch. in an amount of 1 wt. % to 50 wt. %.

In certain embodiments, the preparations comprise:

(i) the preparation of the grassy stem of Ephedra sinica Stapf in an amount of 2 g to 20 g;
(ii) Gypsum ore (CaSO₄.2H₂O) in an amount of 15 g to 60 g;
(iii) the preparation of the seed (nut) of Semen Armeniacae Amarum in an amount of 5 g to 20 g; and/or
(iv) the preparation of the dried root and rhizome of Glycyrrhiza uralensis Fisch. in an amount of 3 g to 20 g.

In certain embodiments, the preparations comprise:

(i) the preparation of the grassy stem of Ephedra sinica Stapf in an amount of 10 g;
(ii) Gypsum ore (CaSO₄.2H₂O) in an amount of 30 g;
(iii) the preparation of the seed (nut) of Semen Armeniacae Amarum in an amount of 10 g; and/or
(iv) the preparation of the dried root and rhizome of Glycyrrhiza uralensis Fisch. in an amount of 10 g.

In certain embodiments, the preparations further comprise:

(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

In certain embodiments, the preparations comprise

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %.

In certain embodiments, the preparations comprise

(v) the preparation of the seed kernel of Coix lacryma-jobi L. var. mayuen (Roman.) Stapf in an amount of 9 g to 30 g;
(vi) the preparation of the dried sclerotia of Poria cocos (Schw.) Wolf in an amount of 5 g to 35 g and
(vii) the preparation of the dried root of Pueraria lobata Ohwi in an amount of 5 g to 35 g.

In certain embodiments, the preparations comprise:

(v) the preparation of the seed kernel of Coix lacryma-jobi L. var. mayuen (Roman.) Stapf in an amount of 15 g;
(vi) the preparation of the dried sclerotia of Poria cocos (Schw.) Wolf in an amount of 20 g and
(vii) the preparation of the dried root of Pueraria lobata Ohwi in an amount of 20 g.

In certain embodiments, provided herein is a pharmaceutical composition obtainable by the method provided herein.

In certain embodiments, provided herein is a method of preventing, treating or managing a respiratory disease and/or a viral infection, comprising administering to a subject in need of such prevention, treatment, or management a pharmaceutical composition provided herein.

In certain embodiments, provided herein is a method of preventing, treating or managing an influenza virus infection or COVID-19, comprising administering to a subject in need of such prevention, treatment, or management a pharmaceutical composition produced from one or more preparations comprising:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %, and
wherein the pharmaceutical composition comprises a pharmaceutically acceptable carrier.

### 3.1 Definitions

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, and unless otherwise specified, the term “subject” or “patient” refers to an animal, including, but not limited to, a mammal, including a primate (e.g., human), cow, sheep, goat, horse, dog, cat, rabbit, rat, bat, pig, or mouse, and a bird, including chicken, duck and turkey. The terms “subject” and “patient” are used interchangeably herein in reference, for example, to a mammalian subject, such as a human subject.

As used herein and unless otherwise indicated, the term “preparation” means a composition prepared from plant part(s) through post-harvest processing, also called primary processing. In certain embodiments, the post-harvest processing includes procedures accorded to plant part obtained from cultivation or field collection to free them from foreign matter, untargeted or extraneous plant materials and other contaminants. In certain embodiments, such procedures include but are not limited to single procedure or combined procedures of inspection, sorting, washing, disinfection, primary cutting, cooling, freezing and/or drying. Additional procedures include but are not limited to single procedure or combined procedures of advanced cutting, comminution (fragmentation), ageing, sweating (fermentation), baking/roasting, boiling/steaming, stir-frying and/or primary distillation. More details on the methods of producing the preparations of plant parts are described in Section 5.3.1. In certain embodiments, the pharmaceutical compositions provided herein are produced from one or more preparations of plant parts. Common procedures include but are not limited to extraction, distillation, fractionation, concentration, fermentation, or other physicochemical or biological methods. In certain embodiments, the resulting pharmaceutical compositions are in the forms of extracts, decoctions, tinctures, essential oils and others. More details on the methods of producing the pharmaceutical compositions from the preparations are described in Section 5.3.2.

As used herein, the term “pharmaceutical composition” refers to the combination of an active agent with a carrier, inert or active, making the composition especially suitable for diagnostic or therapeutic or prophylactic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents and encapsulating agents. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. [1975].

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “prevent,” “preventing” or “prevention” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “manage,” “managing” or “management” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with an agent (e.g., a protein described herein) means an amount capable of treating, preventing, or managing a disorder, disease or condition, or symptoms thereof.

As used herein and unless otherwise indicated, the term “subject” or “patient” includes an organism, including, but not limited to, an animal such a cow, monkey, horse, sheep, pig, chicken, turkey, quail, cat, dog, mouse, rat, rabbit or guinea pig. In one embodiment, the subject or patient is a mammal. In certain embodiments, the subject or patient is a non-human primate. In another embodiment, the subject or patient is a human.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof.

As is known to those of skill in the art, “salts” of the compounds of the present invention may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N dibenzylethylenediamine, chloroprocaine, choline, diethanolamine, ethylenediamine, meglumaine (N-methylglucamine), lysine, and procaine. 3.2 Conventions and Abbreviations

| Abbreviation | Convention |
|---|---|
| COVID-19 | coronavirus disease 2019 |
| CT | computed tomography |
| CPE | cytopathogenic effect |
| ECMO | Extracorporeal Membrane Oxygenation |
| GHPP | good herbal processing practices |
| RT-PCR | Reverse transcription polymerase chain reaction |
| SARS-CoV-2 | severe acute respiratory syndrome coronavirus 2 |
| MDCK cell | Madin-Darby Canine Kidney cell |
| TCM | Traditional Chinese Medicine |
| WHO | World Health Organization |

### 4. BRIEF DESCRIPTION OF THE FIGURES

FIG. 1 illustrates the exemplary Chinese names of the preparations as described herein.

### 5. DETAILED DESCRIPTION OF THE INVENTION

The present application relates to methods of preventing, treating or managing a respiratory disease and/or a viral infection (e.g., an influenza virus infection and COVID-19) using a pharmaceutical composition produced from one or more preparations. The pharmaceutical compositions are described in Section 5.1. The methods of preventing, treating or managing are described in Section 5.2. The methods of producing the pharmaceutical compositions are described in Section 5.3. Specifically, preparations are produced from plant parts, and the pharmaceutical compositions are produced from such preparations. Accordingly, the methods of producing the preparations from the plant parts are described in Section 5.3.1, whereas the methods of producing the pharmaceutical compositions from the preparations of plant parts are described in Section 5.3.2. The assays that can be used to measure the characteristics and/or efficacy of the pharmaceutical compositions are described in Section 5.4.

### 5.1 Pharmaceutical Compositions

In one aspect, provided herein are pharmaceutical compositions. The pharmaceutical compositions as provided herein can be used in the methods of preventing, treating or managing a respiratory disease and/or a viral infection (e.g., an influenza virus infection and COVID-19) as described in Section 5.1. The pharmaceutical compositions as provided herein can be produced using the methods of producing as described in Section 5.3.

5.1.1 Pharmaceutical Compositions Produced from the Preparation(s)

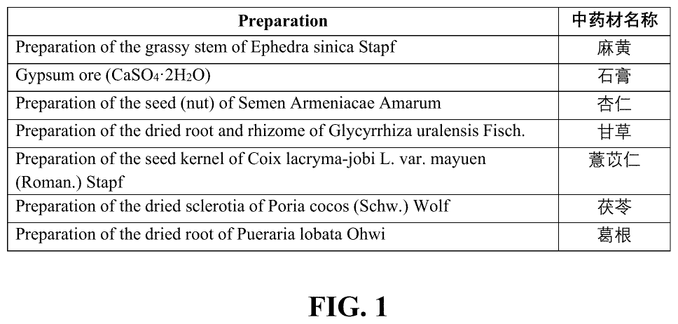

In certain embodiments, a pharmaceutical composition for use with the methods provided herein is produced from one or more preparations of plant parts. A plant is referred to using common Latin names. Such names are well known in the art, for example, see names as provided in Flora of China (Missouri Botanical Garden Press (1999)), Chinese Medicine Dictionary (2nd edition, Editor, NAN JING ZHONG YI YAO DA XUE, Shanghai Science and Technology Press, China (1979)), and National Herbal Compendium (People's Health Publishing House, China (2014)). Exemplary Chinese names of the preparations are listed in FIG. 1.

In certain embodiments, a pharmaceutical composition for use with the methods provided herein is produced from one or more preparations comprising:

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch.

In certain embodiments, the one or more preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %; and/or
(iv) the preparation of the dried root and rhizome of Glycyrrhiza uralensis Fisch. in an amount of 1 wt. % to 50 wt. %.

In certain embodiments, the one or more preparations comprise the preparation of the grassy stem of Ephedra sinica Stapf in an amount of 2 wt. % to 40 wt. %. In certain embodiments, the one or more preparations comprise the preparation the grassy stem of Ephedra sinica Stapf in an amount of 2.5 wt. % to 35 wt. %. In certain embodiments, the one or more preparations comprise the preparation of the grassy stem of Ephedra sinica Stapf in an amount of 3 wt. % to 30 wt. %. In certain embodiments, the one or more preparations comprise the preparation of the grassy stem of Ephedra sinica Stapf in an amount of 4 wt. % to 25 wt. %. In certain embodiments, the one or more preparations comprise the preparation of the grassy stem of Ephedra sinica Stapf in an amount of 5 wt. % to 20 wt. %. In certain embodiments, the one or more preparations comprise the preparation of the grassy stem of Ephedra sinica Stapf in an amount of 6 wt. % to 18 wt. %.

In certain embodiments, the one or more preparations comprise Gypsum ore (CaSO₄.2H₂O) in an amount of 8 wt. % to 75 wt. %. In certain embodiments, the one or more preparations comprise Gypsum ore (CaSO₄.2H₂O) in an amount of 10 wt. % to 70 wt. %. In certain embodiments, the one or more preparations comprise Gypsum ore (CaSO₄.2H₂O) in an amount of 12 wt. % to 65 wt. %. In certain embodiments, the one or more preparations comprise Gypsum ore (CaSO₄.2H₂O) in an amount of 15 wt. % to 60 wt. %. In certain embodiments, the one or more preparations comprise Gypsum ore (CaSO₄.2H₂O) in an amount of 18 wt. % to 55 wt. %. In certain embodiments, the one or more preparations comprise Gypsum ore (CaSO₄.2H₂O) in an amount of 20 wt. % to 55 wt. %.

In certain embodiments, the one or more preparations comprise the preparation of the seed (nut) of Semen Armeniacae Amarum in an amount of 2.5 wt. % to 40 wt. %. In certain embodiments, the one or more preparations the preparation of the seed (nut) of Semen Armeniacae Amarum in an amount of 2.5 wt. % to 35 wt. %. In certain embodiments, the one or more preparations comprise the preparation of the seed (nut) of Semen Armeniacae Amarum in an amount of 3 wt. % to 30 wt. %. In certain embodiments, the one or more preparations comprise the preparation of the seed (nut) of Semen Armeniacae Amarum in an amount of 4 wt. % to 25 wt. %. In certain embodiments, the one or more preparations comprise the preparation of the seed (nut) of Semen Armeniacae Amarum in an amount of 5 wt. % to 20 wt. %. In certain embodiments, the one or more preparations comprise the preparation of the seed (nut) of Semen Armeniacae Amarum in an amount of 6 wt. % to 18 wt. %.

In certain embodiments, the one or more preparations comprise the preparation of the dried root and rhizome of Glycyrrhiza uralensis Fisch. in an amount of 2 wt. % to 40 wt. %. In certain embodiments, the one or more preparations comprise the preparation of the dried root and rhizome of Glycyrrhiza uralensis Fisch. in an amount of 2.5 wt. % to 35 wt. %. In certain embodiments, the one or more preparations comprise the preparation of the dried root and rhizome of Glycyrrhiza uralensis Fisch. in an amount of 3 wt. % to 30 wt. %. In certain embodiments, the one or more preparations comprise the preparation of the dried root and rhizome of Glycyrrhiza uralensis Fisch. in an amount of 4 wt. % to 25 wt. %. In certain embodiments, the one or more preparations comprise the preparation of the dried root and rhizome of Glycyrrhiza uralensis Fisch. in an amount of 5 wt. % to 20 wt. %. In certain embodiments, the one or more preparations comprise the preparation of the dried root and rhizome of Glycyrrhiza uralensis Fisch. in an amount of 6 wt. % to 18 wt. %.

In certain embodiments, the one or more preparations comprise

(i) the preparation of the grassy stem of Ephedra sinica Stapf in an amount of 2 g to 20 g;
(ii) Gypsum ore (CaSO₄.2H₂O) in an amount of 15 g to 60 g;
(iii) the preparation of the seed (nut) of Semen Armeniacae Amarum in an amount of 5 g to 20 g; and/or
(iv) the preparation of the dried root and rhizome of Glycyrrhiza uralensis Fisch. in an amount of 3 g to 20 g.

In certain embodiments, the one or more preparations comprise the preparation of the grassy stem of Ephedra sinica Stapf in an amount of 3 g to 18 g. In certain embodiments, the one or more preparations comprise the preparation the grassy stem of Ephedra sinica Stapf in an amount of 5 g to 15 g. In certain embodiments, the one or more preparations comprise the preparation of the grassy stem of Ephedra sinica Stapf in an amount of 6 g to 14 g. In certain embodiments, the one or more preparations comprise the preparation of the grassy stem of Ephedra sinica Stapf in an amount of 7 g to 13 g. In certain embodiments, the one or more preparations comprise the preparation of the grassy stem of Ephedra sinica Stapf in an amount of 8 g to 12 g. In certain embodiments, the one or more preparations comprise the preparation of the grassy stem of Ephedra sinica Stapf in an amount of 10 g.

In certain embodiments, the one or more preparations comprise Gypsum ore (CaSO₄.2H₂O) in an amount of 18 g to 50 g. In certain embodiments, the one or more preparations comprise Gypsum ore (CaSO₄.2H₂O) in an amount of 20 g to 45 g. In certain embodiments, the one or more preparations comprise Gypsum ore (CaSO₄.2H₂O) in an amount of 22 g to 40 g. In certain embodiments, the one or more preparations comprise Gypsum ore (CaSO₄.2H₂O) in an amount of 25 g to 35 g. In certain embodiments, the one or more preparations comprise Gypsum ore (CaSO₄.2H₂O) in an amount of 28 g to 32 g. In certain embodiments, the one or more preparations comprise Gypsum ore (CaSO₄.2H₂O) in an amount of 30 g.

In certain embodiments, the one or more preparations comprise the preparation of the seed (nut) of Semen Armeniacae Amarum in an amount of 6 g to 18 g. In certain embodiments, the one or more preparations the preparation of the seed (nut) of Semen Armeniacae Amarum in an amount of 7 g to 16 g. In certain embodiments, the one or more preparations comprise the preparation of the seed (nut) of Semen Armeniacae Amarum in an amount of 8 g to 14 g. In certain embodiments, the one or more preparations comprise the preparation of the seed (nut) of Semen Armeniacae Amarum in an amount of 9 g to 12 g. In certain embodiments, the one or more preparations comprise the preparation of the seed (nut) of Semen Armeniacae Amarum in an amount of 10 g.

In certain embodiments, the one or more preparations comprise the preparation of the dried root and rhizome of Glycyrrhiza uralensis Fisch. in an amount of 4 g to 18 g. In certain embodiments, the one or more preparations comprise the preparation of the dried root and rhizome of Glycyrrhiza uralensis Fisch. in an amount of 5 g to 16 g. In certain embodiments, the one or more preparations comprise the preparation of the dried root and rhizome of Glycyrrhiza uralensis Fisch. in an amount of 6 g to 14 g. In certain embodiments, the one or more preparations comprise the preparation of the dried root and rhizome of Glycyrrhiza uralensis Fisch. in an amount of 7 g to 12 g. In certain embodiments, the one or more preparations comprise the preparation of the dried root and rhizome of Glycyrrhiza uralensis Fisch. in an amount of 8 g to 12 g. In certain embodiments, the one or more preparations comprise the preparation of the dried root and rhizome of Glycyrrhiza uralensis Fisch. in an amount of 10 g.

In certain embodiments, the one or more preparations comprise:

(i) the preparation of the grassy stem of Ephedra sinica Stapf in an amount of 10 g;
(ii) Gypsum ore (CaSO₄.2H₂O) in an amount of 30 g;
(iii) the preparation of the seed (nut) of Semen Armeniacae Amarum in an amount of 10 g; and/or
(iv) the preparation of the dried root and rhizome of Glycyrrhiza uralensis Fisch. in an amount of 10 g.

In certain embodiments, the one or more preparations further comprise:

(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

In certain embodiments, the one or more preparations comprise:

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the one or more preparations comprise:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %.

In certain embodiments, the one or more preparations comprise the preparation of the seed kernel of Coix lacryma-jobi L. var. mayuen (Roman.) Stapf in an amount of 5 wt. % to 40 wt. %. In certain embodiments, the one or more preparations comprise the preparation the seed kernel of Coix lacryma-jobi L. var. mayuen (Roman.) Stapf in an amount of 7 wt. % to 35 wt. %. In certain embodiments, the one or more preparations comprise the preparation of the seed kernel of Coix lacryma-jobi L. var. mayuen (Roman.) Stapf in an amount of 8 wt. % to 30 wt. %. In certain embodiments, the one or more preparations comprise the preparation of the seed kernel of Coix lacryma-jobi L. var. mayuen (Roman.) Stapf in an amount of 10 wt. % to 25 wt. %. In certain embodiments, the one or more preparations comprise the preparation of the seed kernel of Coix lacryma-jobi L. var. mayuen (Roman.) Stapf in an amount of 12 wt. % to 20 wt. %. In certain embodiments, the one or more preparations comprise the preparation of the seed kernel of Coix lacryma-jobi L. var. mayuen (Roman.) Stapf in an amount of 12 wt. % to 18 wt. %.

In certain embodiments, the one or more preparations comprise the preparation of the dried sclerotia of Poria cocos (Schw.) Wolf in an amount of 2 wt. % to 50 wt. %. In certain embodiments, the one or more preparations comprise the preparation of the dried sclerotia of Poria cocos (Schw.) Wolf in an amount of 3 wt. % to 40 wt. %. In certain embodiments, the one or more preparations comprise the preparation of the dried sclerotia of Poria cocos (Schw.) Wolf in an amount of 3 wt. % to 30 wt. %. In certain embodiments, the one or more preparations comprise the preparation of the dried sclerotia of Poria cocos (Schw.) Wolf in an amount of 4 wt. % to 25 wt. %. In certain embodiments, the one or more preparations comprise the preparation of the dried sclerotia of Poria cocos (Schw.) Wolf in an amount of 5 wt. % to 20 wt. %. In certain embodiments, the one or more preparations comprise the preparation of the dried sclerotia of Poria cocos (Schw.) Wolf in an amount of 10 wt. % to 18 wt. %.

In certain embodiments, the one or more preparations comprise the preparation of the dried root of Pueraria lobata Ohwi in an amount of 2 wt. % to 50 wt. %. In certain embodiments, the one or more preparations comprise the preparation of the dried root of Pueraria lobata Ohwi in an amount of 3 wt. % to 40 wt. %. In certain embodiments, the one or more preparations comprise the preparation of the dried root of Pueraria lobata Ohwi in an amount of 3 wt. % to 30 wt. %. In certain embodiments, the one or more preparations comprise the preparation of the dried root of Pueraria lobata Ohwi in an amount of 4 wt. % to 25 wt. %. In certain embodiments, the one or more preparations comprise the preparation of the dried root of Pueraria lobata Ohwi in an amount of 5 wt. % to 20 wt. %. In certain embodiments, the one or more preparations comprise the preparation of the dried root of Pueraria lobata Ohwi in an amount of 10 wt. % to 18 wt. %.

In certain embodiments, the one or more preparations comprise

(v) the preparation of the seed kernel of Coix lacryma-jobi L. var. mayuen (Roman.) Stapf in an amount of 9 g to 30 g;
(vi) the preparation of the dried sclerotia of Poria cocos (Schw.) Wolf in an amount of 5 g to 35 g and
(vii) the preparation of the dried root of Pueraria lobata Ohwi in an amount of 5 g to 35 g.

In certain embodiments, the one or more preparations comprise the preparation of the seed kernel of Coix lacryma-jobi L. var. mayuen (Roman.) Stapf in an amount of 6 g to 30 g. In certain embodiments, the one or more preparations comprise the preparation of the seed kernel of Coix lacryma-jobi L. var. mayuen (Roman.) Stapf in an amount of 8 g to 28 g. In certain embodiments, the one or more preparations comprise the preparation of the seed kernel of Coix lacryma-jobi L. var. mayuen (Roman.) Stapf in an amount of 10 g to 25 g. In certain embodiments, the one or more preparations comprise the preparation of the seed kernel of Coix lacryma-jobi L. var. mayuen (Roman.) Stapf in an amount of 15 g to 22 g. In certain embodiments, the one or more preparations comprise the preparation of the seed kernel of Coix lacryma-jobi L. var. mayuen (Roman.) Stapf in an amount of 18 g to 22 g. In certain embodiments, the one or more preparations comprise the preparation of the seed kernel of Coix lacryma-jobi L. var. mayuen (Roman.) Stapf in an amount of 20 g.

In certain embodiments, the one or more preparations comprise the preparation of the dried sclerotia of Poria cocos (Schw.) Wolf in an amount of 6 g to 30 g. In certain embodiments, the one or more preparations comprise the preparation the dried sclerotia of Poria cocos (Schw.) Wolf in an amount of 8 g to 28 g. In certain embodiments, the one or more preparations comprise the preparation of the dried sclerotia of Poria cocos (Schw.) Wolf in an amount of 9 g to 25 g. In certain embodiments, the one or more preparations comprise the preparation of the dried sclerotia of Poria cocos (Schw.) Wolf in an amount of 10 g to 22 g. In certain embodiments, the one or more preparations comprise the preparation of the dried sclerotia of Poria cocos (Schw.) Wolf in an amount of 15 g to 22 g. In certain embodiments, the one or more preparations comprise the preparation of the dried sclerotia of Poria cocos (Schw.) Wolf in an amount of 20 g.

In certain embodiments, the one or more preparations comprise the preparation of the dried root of Pueraria lobata Ohwi in an amount of 6 g to 30 g. In certain embodiments, the one or more preparations comprise the preparation of the dried root of Pueraria lobata Ohwi an amount of 8 g to 28 g. In certain embodiments, the one or more preparations comprise the preparation of the dried root of Pueraria lobata Ohwi in an amount of 9 g to 25 g. In certain embodiments, the one or more preparations comprise the preparation of the dried root of Pueraria lobata Ohwi in an amount of 10 g to 22 g. In certain embodiments, the one or more preparations comprise the preparation of the dried root of Pueraria lobata Ohwi in an amount of 15 g to 22 g. In certain embodiments, the one or more preparations comprise the preparation of the dried root of Pueraria lobata Ohwi in an amount of 20 g.

In certain embodiments, the one or more preparations comprise:

(v) the preparation of the seed kernel of Coix lacryma-jobi L. var. mayuen (Roman.) Stapf in an amount of 15 g;
(vi) the preparation of the dried sclerotia of Poria cocos (Schw.) Wolf in an amount of 20 g and
(vii) the preparation of the dried root of Pueraria lobata Ohwi in an amount of 20 g.

In certain embodiments, the one or more preparations are any two preparations selected from the group consisting of

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the one or more preparations are any three preparations selected from the group consisting of

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the one or more preparations are any four preparations selected from the group consisting of

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the one or more preparations are any five preparations selected from the group consisting of

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the one or more preparations are any six preparations selected from the group consisting of

(i) a preparation of the grassy stem of Ephedra sinica Stapf;
(ii) Gypsum ore (CaSO₄.2H₂O);
(iii) a preparation of the seed (nut) of Semen Armeniacae Amarum;
(iv) a preparation of the dried root and rhizome of Glycyrrhiza uralensis Fisch.;
(v) a preparation of the seed kernel of Coix lacryma-jobi L. var. mayuen (Roman.) Stapf;
(vi) a preparation of the dried sclerotia of Poria cocos (Schw.) Wolf; and
(vii) a preparation of the dried root of Pueraria lobata Ohwi.

In certain embodiments, the pharmaceutical compositions as provided herein may be modified by including other preparations in production. Such modification may be apparent to those skilled in the art, for example, due to the fact that such other preparations have been used to treat related diseases.

5.1.2 Dosage Forms

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water.

In certain embodiments, the pharmaceutical composition as described herein comprises water as one pharmaceutically acceptable carrier. In certain embodiments, the pharmaceutical composition as described herein comprises β-cyclodextrin as one pharmaceutically acceptable carrier.

(i) Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., decoctions). Such dosage forms contain predetermined amounts of active ingredients and may be prepared by some known methods of pharmacy. See generally, Remington's Pharmaceutical Sciences, 18th ed., Mack Publishing, Easton Pa. (1990).

In certain embodiments, the oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules may represent advantageous oral dosage unit forms, in which case solid excipients are employed. If desired, tablets can be coated by standard aqueous or non-aqueous techniques. Such dosage forms may be prepared by some known methods of pharmacy. In certain embodiments, pharmaceutical compositions and dosage forms are prepared by uniformly and intimately admixing the active ingredients with liquid carriers, finely divided solid carriers, or both, and then shaping the product into the desired presentation if necessary.

In certain embodiments, a tablet is prepared by compression or molding. In certain embodiments, compressed tablets are be prepared by compressing in a suitable machine the active ingredients in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, AVICEL-PH-101, AVICEL-PH-103 AVICEL RC-581, AVICEL-PH-105 (FMC Corporation, American Viscose Division, Avicel Sales, Marcus Hook, Pa.), and mixtures thereof. An specific binder is a mixture of microcrystalline cellulose and sodium carboxymethyl cellulose (e.g., AVICEL RC-581). Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant.

Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200, W.R. Grace Co., Baltimore, Md.), a coagulated aerosol of synthetic silica (Degussa Co. of Plano, Tex.), CAB-O-SIL (a pyrogenic silicon dioxide, Cabot Co. of Boston, Mass.), and mixtures thereof. In certain embodiments, if used at all, lubricants are used in an amount of less than about 1 weight percent of the pharmaceutical compositions or dosage forms into which they are incorporated.

(ii) Delayed Release Dosage Forms

In certain embodiments, the active ingredients provided herein are administered by controlled release means or by delivery devices. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated herein by reference in its entirety. In certain embodiments, such dosage forms are be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic or prophylactic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

(iii) Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms provided herein. For example, cyclodextrin and its derivatives can be used to increase the solubility of Compound A, or an enantiomer or a mixture of enantiomers thereof, or a pharmaceutically acceptable salt, solvate, hydrate, co-crystal, clathrate, or polymorph thereof. See, e.g., U.S. Pat. No. 5,134,127, the disclosure of which is incorporated herein by reference in its entirety.

(iv) Topical and Mucosal Dosage Forms

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein depend on the particular tissue to which a given pharmaceutical composition or dosage form will be applied. With that fact in mind, in certain embodiments, the excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Additional examples of such ingredients can be found, e.g., in Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 5.2 Methods of Preventing, Treating or Managing

In one aspect, the present application provides methods of preventing, treating or managing a respiratory disease and/or a viral infection (e.g., an influenza virus infection and COVID-19) using a pharmaceutical composition produced as described in Section 5.1. The diseases are described in Section 5.2.1. Doses and administrations are described in Section 5.2.2. Combination therapies are described in Section 5.2.3.

In certain embodiments, provided herein is a method of preventing, treating or managing a respiratory disease and/or a viral infection, comprising administering to a subject in need of such prevention, treatment, or management a pharmaceutical composition provided herein.

In certain embodiments, provided herein is a method of preventing, treating or managing a respiratory disease and/or a viral infection, comprising administering to a subject in need of such prevention, treatment, or management a pharmaceutical composition produced from one or more preparations comprising

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the respiratory disease is pneumonia.

In certain embodiments, the respiratory disease and/or the viral infection is an influenza virus infection. In certain embodiments, the influenza virus is influenza virus A.

In certain embodiments, the respiratory disease and/or the viral infection is a coronavirus infection. In certain embodiments, the coronavirus is the severe acute respiratory syndrome coronavirus 2 (SARS-CoV-2). In certain embodiments, the coronavirus infection is coronavirus disease 2019 (COVID-19).

In certain embodiments, the subject has symptoms comprising fever (body temperature at about 99.5° F. or above), dry cough, and/or difficulty breathing. In certain embodiments, the subject shows abnormality in a chest computed tomography (CT) scan.

In certain embodiments, the method treats and/or manages the respiratory disease and/or the viral infection.

In certain embodiments, provided herein is a method of preventing, treating or managing COVID-19, comprising administering to a subject in need of such prevention, treatment, or management a pharmaceutical composition produced from one or more preparations comprising:

(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the method treats and/or manages COVID-19.

In certain embodiments, the method further comprises administering to the subject a second agent. In certain embodiments, the second agent is an antiviral agent, an anti-inflammatory agent, a Janus kinase inhibitor, a steroid hormone, an immunosuppressive agent, an agent targeting interleukin-6 receptor, an antibacterial agent, an anti-parasitic agent, Traditional Chinese medicine, stem cells, convalescent plasma, or a combination thereof. In certain embodiments, the second agent is remdesivir, LPV/r (lopinivir-ritonavir), arbidol, favipiravir, baricitinib, corticosteroids, interferon, tocilizumab, sarilumab, azithromycin, hydroxychloroquine or chloroquine, or a combination thereof.

In certain embodiments, the pharmaceutical composition is administered via oral route.

In certain embodiments, provided herein is a method of preventing, treating or managing an influenza virus infection or COVID-19, comprising administering to a subject in need of such prevention, treatment, or management a pharmaceutical composition produced from one or more preparations comprising:

(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %, and
wherein the pharmaceutical composition comprises a pharmaceutically acceptable carrier.

In certain embodiments, the methods provided herein treat a respiratory disease and/or a viral infection in a subject. In certain embodiments, the methods provided herein manage a respiratory disease and/or a viral infection in a subject. In certain embodiments, the methods provided herein prevent a respiratory disease and/or a viral infection in a subject.

In certain embodiments, the methods may be used to treat or manage a respiratory disease and/or a viral infection in a subject who has been diagnosed with the respiratory disease and/or the viral infection. In certain embodiments, the subject has been diagnosed using methods known in the art, for example, a nucleic-acid based detection method. In certain embodiments, the methods may be used to treat or manage a respiratory disease and/or a viral infection in a subject who has been suspected to have the respiratory disease and/or the viral infection, for example, due to possible exposure and/or showing of symptoms. In certain embodiments, the methods may be used to treat or manage a respiratory disease and/or a viral infection in a subject who has shown no obvious symptom from the respiratory disease and/or the viral infection.

In certain embodiments, the methods may be used to prevent a respiratory disease and/or a viral infection in a subject who has never been diagnosed with the respiratory disease and/or the viral infection. In certain embodiments, the methods may be used to prevent a respiratory disease and/or a viral infection reoccurring in a subject who has been diagnosed with the respiratory disease and/or the viral infection previously. In certain embodiments, the methods may be used to prevent a respiratory disease and/or a viral infection in a subject who has been exposed to the viral infection. In certain embodiments, the methods may be used to prevent a respiratory disease and/or a viral infection in a subject who is a health care working exposed to the viral infection.

In certain embodiments, the methods result in less number of subject being hospitalized due to the diseases. In certain embodiments, the methods result in improvement in symptoms due to the diseases. In certain embodiments, the methods result in less number of subject having severe symptoms due to the diseases. In certain embodiments, the methods result in less number of subject having respiratory failure due to the diseases. In certain embodiments, the methods result in a change from baseline (for example, pre-dose) viral load (for example, SAR-CoV-2 load) from patient samples (for example, nasopharyngeal samples) on certain days post starting of treatment (for example, 3, 4, 5, 6, 7, 8, 9, 10, 11, 12, 13, and 14 days post starting of treatment). In certain embodiments, the methods result in a difference between the treatment group and the control group (for example, placebo group) in the viral load AUC (Area under the Curve) during certain days (for example, days 1-3; days 1-4, days 1-5, days 1-6, days 1-7, days 1-8, days 1-9, days 1-10, days 1-11, days 1-12, days 1-13, and days 1-14). In certain embodiments, the viral load is from patient nasopharyngeal samples. In certain embodiments, the viral load is measured by qRT-PCR.

In certain embodiments, the methods result in improvement in the odds of ratio for improvement on a 7-point ordinal Scale on certain day post starting treatment, for example 14 days. In certain embodiments, the odds ratio represents the odds of improvement in the ordinal scale between the treatment groups. In certain embodiments, the ordinal scale is an assessment of the clinical status at a given day. Each day, the worst score from the previous day will be recorded. In certain embodiments, the scale is as follows: 1. Death; 2. Hospitalized, on invasive mechanical ventilation or Extracorporeal Membrane Oxygenation (ECMO); 3. Hospitalized, on non-invasive ventilation or high flow oxygen devices; 4. Hospitalized, requiring low flow supplemental oxygen; 5. Hospitalized, not requiring supplemental oxygen—requiring ongoing medical care (disease related or otherwise); 6. Hospitalized, not requiring supplemental oxygen—no longer required ongoing medical care besides the methods of treatment described herein; 7. Not hospitalized.

In certain embodiments, the methods reduce incidence of the disease. In certain embodiments, the methods provided herein prevent a respiratory disease and/or a viral infection symptoms as recorded in a daily diary. In certain embodiments, the methods lead to higher proportion of participants with no evidence of viral infection in comparison to no treatment.

5.2.1 Diseases

In one aspect, the disease to be prevented, treated or managed by the methods provided herein is a respiratory disease. In certain embodiments, the respiratory disease affects the bronchus and lungs. Exemplary respiratory diseases include but are not limited to an obstructive lung disease, reversible airways disease, asthma, chronic obstructive pulmonary disease (COPD), emphysema, bronchitis, Ondine's curse, lung cancer, tuberculosis or a lung disease where shortness of breath as a chronic symptom. In certain embodiments, the respiratory disease is pneumonia.

In certain embodiments, the respiratory disease is caused by an infection caused a pathogen. Exemplary types of pathogens include but are not limited to virus, bacteria, fungi and yeast. In certain embodiments, the respiratory disease is caused by a viral infection. In certain embodiment, the disease causes epidemics or pandemics.

In another aspect, the disease to be prevented, treated or managed by the methods provided herein is a viral infection. Exemplary types of virus include but are not limited to rhinovirus, respiratory syncytial virus, influenza virus, parainfluenza virus, human metapneumovirus, measles, mumps, adenovirus, and coronavirus. In certain embodiments, the viral infection causes respiratory symptoms, for example, cough, sore throat, wheezing, chest tightness, difficulty with exertion, shortness of breath, sore throat. In certain embodiments, the viral infection causes other symptoms, for example, gastrointestinal (e.g., nausea and diarrhea) and/or neurologic symptoms (e.g., anosmia, ageusia, and headache), difficulty speaking, dry mouth, and malaise.

(i) Influenza Virus Infection

In one aspect, the disease to be prevented, treated or managed by the methods provided herein is an influenza virus infection.

In certain embodiments, the influenza virus infection is caused by influenza A, B, C or D virus and subtypes thereof, or any other types of influenza viruses. In certain embodiments, the influenza virus infection is a seasonal flu. In certain embodiments, the seasonal flu is caused by influenza A or B virus.

In certain embodiments, the influenza virus infection is caused by influenza A virus and subtypes thereof. Exemplary influenza A virus subtypes include but are not limited to H1N1, H2N2, H3N2, H5N1, H7N9, H7N7, H1N2, H9N2, H7N2, H7N3, H5N2, and H10N7.

In certain embodiments, the influenza virus infects a human subject. In certain embodiments, the influenza virus infects a subject other than humans, for example, a mammal, including a primate (e.g., human), cow, sheep, goat, horse, dog, cat, rabbit, rat, bat, pig or mouse, and a bird, including chicken, duck and turkey. In certain embodiments, the influenza virus is capable of infecting a human subject and one or more other species, including the animals mentioned above.

(ii) COVID-19

In another aspect, the disease to be prevented, treated or managed by the methods provided herein is coronavirus disease 2019 (COVID-19). In certain embodiments, the COVID-19 is caused by severe acute respiratory syndrome coronavirus 2 (SARS-CoV-2). In yet another aspect, the disease to be prevented, treated or managed by the methods provided herein is a coronavirus infection, for example, an SARS-CoV-2 infection.

In certain embodiments, COVID-19 is diagnosed by detection of SARS-CoV-2 in subject samples. In certain embodiments, the detection is nucleic-acid based. In certain embodiments, COVID-19 is diagnosed by detection of RNA from SARS-CoV-2 in upper and/or lower respiratory specimens.

In certain embodiments, a subject having COVID-19 is asymptomatic.

In certain embodiments, a subject having COVID-19 has mild symptoms. In certain embodiments, a subject having COVID-19 has a body temperature below about 99.5° F. In certain embodiments, a subject having COVID-19 has one or more symptoms comprising cough, sore throat, and/or new loss of taste or smell.

In certain embodiments, a subject having COVID-19 has severe symptoms. In certain embodiments, a subject having COVID-19 has a body temperature at about 99.5° F. or above. In certain embodiments, a subject having COVID-19 has one or more symptoms comprising abnormality in a chest computed tomography (CT) scan, dry cough and/or difficulty breathing.

In certain embodiments, the SARS-CoV-2 infects a human subject. In certain embodiments, the SARS-CoV-2 infects a subject other than humans, for example, a mammal, including a primate (e.g., human), cow, sheep, goat, horse, dog, cat, rabbit, rat, bat, pig or mouse, and a bird, including chicken, duck and turkey. In certain embodiments, the SARS-CoV-2 is capable of infecting a human subject and one or more other species, including the animals mentioned above.

5.2.2 Doses and Administration

The amount of the pharmaceutical composition as described herein may be selected in order to achieve a desired therapeutic or prophylactic effect. The amount of the pharmaceutical composition may be selected in order to achieve a desired therapeutic or prophylactic effect.

In certain embodiments, the pharmaceutical composition as described herein is administered in a therapeutically or prophylactically effective amount. In certain embodiments, the pharmaceutical composition as described herein is administered via oral, intravenous, intraarterial, intraperitoneal, intramuscular, subcutaneous, intrapleural, intrathecal, or intracavitary route, or by perfusion through a catheter or by direct intralesional injection.

In certain embodiments, the pharmaceutical composition is administered once or more times daily, every other day, weekly, bi-weekly, monthly or yearly, or even once every 2 to 20 years. In certain embodiments, the pharmaceutical composition is administered daily. In certain embodiments, the pharmaceutical composition is administered twice daily.

In certain embodiments, the pharmaceutical composition administered daily (for example, twice a day) is described in Section 5.1. In certain embodiments, the pharmaceutical composition administered daily (for example, twice a day) is produced from one or more preparations in amounts that are different from the amounts described in Section 5.1, for example, due to different production parameters and/or methods, yet the pharmaceutical composition comprises the same amount of active ingredients as that produced from the preparations in the amounts described in Section 5.1. In certain embodiments, the amount of the preparations as described herein may be selected in order to achieve a desired therapeutic or prophylactic effect.

5.2.3 Combination Therapies

In certain embodiments, the methods provided herein further comprise administering to the patient a second agent.

In certain embodiments, the second agent is an antiviral agent, an anti-inflammatory agent, a Janus kinase inhibitor, a steroid hormone, an immunosuppressive agent, an agent targeting interleukin-6 receptor, an antibacterial agent, an anti-parasitic agent, a pain reliever, Traditional Chinese medicine, stem cells, convalescent plasma, or a combination thereof.

In certain embodiments, the second agent is an anti-HIV agent that treats, manages, or prevents HIV infection. Exemplary anti-HIV agents include but are not limited to abacavir, didanosine, emtricitabine, lamivudine, stavudine, tenofovir alafenamide, tenofovir disoproxil fumarate, zidovudine, delavirdine, doravirine, efavirenz, etravirine, nevirapine, rilpivirine, atazanavir, darunavir, fosamprenavir, indinavir, nelfinavir, ritonavir, saquinavir, tipranavir, bictegravir, dolutegravir, elvitegravir, raltegravir, enfuvirtide, maraviroc, ibalizumab-uiyk, and ritonavir (RTV).

In certain embodiments, the second agent is an anti-influenza virus agent that treats, manages, or prevents influenza virus infection. Exemplary anti-influenza virus agents include but are not limited to adamantan(amin)e derivatives, neuraminidase inhibitors, Oseltamivir and zanamivir, GS4071, peramivir and other cyclopentane or pyrrolidine derivatives, dimeric neuraminidase inhibitors, ribavirin and viramidine, sialidase fusion protein and sialylglycopolymers, siRNAs and phosphorothioate oligonucleotides, Influenza-virus RNA-polymerase inhibitors, interferon (inducers), and signal-transduction inhibitors.

In certain embodiments, the second agent is a steroid hormone. Exemplary steroid hormones include but are not limited to mineralocorticoids, glucocorticoids, androgens, estrogens and progestins, transport and fate of steroid hormones.

In certain embodiments, the second agent is an immunosuppressive agent. In certain embodiments, the immunosuppressive agent is a corticosteroid, an mTOR inhibitor, an IMDH inhibitors, a biologic or a Janus kinase inhibitor, or a Calcineurin inhibitor. Exemplary immunosuppressive agents include but are not limited to prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), sirolimus (Rapamune), everolimus (Afinitor, Zortress), azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), and daclizumab (Zinbryta).

In certain embodiments, the second agent is remdesivir, LPV/r (lopinivir-ritonavir), arbidol, favipiravir, baricitinib, corticosteroids, interferon, tocilizumab, sarilumab, azithromycin, chloroquine, ibuprofen, acetaminophen, or a combination thereof.

In certain embodiments, the second agent is medication that improves symptoms, for example, cough suppressants.

In certain embodiments, the second agent is administered according to the locally approved label or Pharmacy manual for preparation, administration, and storage information.

### 5.3 Methods of Producing a Pharmaceutical Composition

In one aspect, provided herein are methods of producing the pharmaceutical compositions as described in Section 5.1. Specifically, preparations are produced from plant parts, and the pharmaceutical compositions are produced from such preparations. Accordingly, the methods of producing the preparations from the plant parts are described in Section 5.3.1, whereas the methods of producing the pharmaceutical compositions from the preparations of plant parts are described in Section 5.3.2.

5.3.1 Method of Producing the Preparations of Plant Parts

In certain embodiments, a preparation is prepared from raw plant parts through a post-harvest processing, also called primary processing. In certain embodiments, the post-harvest processing includes procedures accorded to plant part obtained from cultivation or field collection to free them from foreign matter, untargeted or extraneous plant materials and other contaminants. In certain embodiments, such procedures include but are not limited to single procedure or combined procedures of inspection, sorting, washing, disinfection, primary cutting, cooling, freezing and/or drying. Additional procedures include but are not limited to single procedure or combined procedures of advanced cutting, comminution (fragmentation), ageing, sweating (fermentation), baking/roasting, boiling/steaming, stir-frying and/or primary distillation.

In certain embodiments, a preparation described herein is produced from a plant part using known skills in the art, for example, methods described in WHO guidelines on good herbal processing practices (GHPP) for herbal medicines (WHO Expert Committee on Specifications for Pharmaceutical Preparations, fifty-second report, WHO Technical Report Series, No. 1010 (2018)), Chinese Medicine Dictionary (2^{nd} edition, Editor, NAN JING ZHONG YI YAO DA XUE; Shanghai Science and Technology Press, China (1979)), and editions of Chinese Pharmacopoeia (ChP) (compiled by the Pharmacopoeia Commission of the Ministry of Health of the People's Republic of China, for example 10th edition (2015)). In certain embodiments, the primary processing procedures of a particular plant part is well known in the art. In certain embodiments, the primary processing procedures, quality and characteristics of the resulting preparation is known and accepted in the art, for example, as described in Chinese Medicine Dictionary (2^{nd} edition, Editor, NAN JING ZHONG YI YAO DA XUE; Shanghai Science and Technology Press, China (1979)) and editions of Chinese Pharmacopoeia (ChP) (compiled by the Pharmacopoeia Commission of the Ministry of Health of the People's Republic of China, for example 10^{th} edition (2015)).

(i) Sorting

In certain embodiments, the sorting process serves as the first step of the procedures to generate the preparations from raw plant part. After the bulk amount of the desired plant part has been harvested or collected, all extraneous and unwanted matter including dirt (for example, soil, dust, mud and stones), impurities (for example, insects, rotten tissues, untargeted/extraneous medicinal plant(s) and/or plant part(s)), and residual non-medicinal as well as toxic part(s) must be removed from the medicinal part(s). Depending on the plant part, the process may involve procedures such as: removing dirt and foreign substances; discarding damaged parts; peeling (to separate unwanted plant part(s) from the medicinal plant part(s) such as removing unwanted root bark from the roots or collecting stem bark from the stem); sieving, trimming, singeing (to remove hairs or rootlets); removal of residues of unwanted plant part(s) (for example, removing unwanted seeds from fruits and stripping leaves from stems).

(ii) Washing and Leaching

In certain embodiments, the procedures comprise washing. Raw plant part, especially roots, rhizomes and tubers, can be washed with clean water and dried soon after harvest or collection. During the washing process, scraping and brushing may be necessary.

In certain embodiments, the procedures comprise leaching. Some impurities can be removed by the action of running water over the raw plant part (leaching). The duration of leaching has to be controlled in order to prevent excessive loss of active ingredients.

(iii) Drying

In certain embodiments, the procedures comprise drying. Unless used in the fresh state, the raw plant parts need to be dried after being sorted and washed. In general, they are dried as soon as possible to protect them from mold and other microbial infestation. Drying will also prevent tissue deterioration and phytochemical alteration caused by the actions of enzymes and microbial organisms. It will also facilitate grinding and milling, and converts the plant parts into a convenient form for further processing. However, attention must be given to the potential loss of volatile (for example, essential oil) constituents present in the fresh material. The final moisture content for dried preparations varies depending on the tissue structure, and may be below 12%. Information on the appropriate moisture content for a particular preparation may be available from pharmacopoeias or other monographs. Proper drying involves four major aspects: control of temperature, humidity, airflow and cleanliness of the air. The drying conditions are determined by the nature of the raw medicinal plant material to be dried (tissue structure and chemical composition) and by the desired appearance of the final form. The drying method used may have considerable impact on the quality of the resulting preparations. Hence, the choice of a suitable procedure is crucial. Information on appropriate drying methods and procedures for particular preparations may be available from pharmacopoeias or other authoritative monographs. Raw plant parts are most often dried by sun-drying, shade-drying or by artificial heat. The drying conditions chosen should be appropriate to the type of the plant part. They are dependent on the characteristics (for example, volatility and stability) of the active ingredients and the texture of the plant part collected (for example, root, leaf or flower). Generally, one of the following drying processes can be adopted. Drying may be carried out by sun-drying (under direct sunlight), shade-drying (in the shade with or without artificial airflow to avoid direct exposure to strong sunlight), or drying by artificial heat.

(iv) Other Procedures

In certain embodiments, the procedures comprise primary cutting. Bulky raw plant parts that have been harvested or collected may require primary cutting to reduce their size before transportation to the processing or manufacturing facility. Primary cutting is usually performed at or near the harvest or collection site.

In certain embodiments, the procedures comprise ageing. The ageing process refers to storing the plant part for a period of time after harvesting or collection from the field prior to use. Plant parts can generally be aged in the sun or in the shade, depending on the specific plant part. During the process of ageing, excessive water is evaporated and enzymatic reactions (such as hydrolysis of the glycone portion of glycosides) or oxidation may occur to alter the chemical composition of the plant part.

In certain embodiments, the procedures comprise sweating. Sweating can involve keeping the plant parts at a temperature of 45 to 65° C. in conditions of high humidity for an extended period, from one week to two months, depending on the plant species. The sweating process is considered a hydrolytic and oxidative process in which some of the chemical ingredients within the plant parts are hydrolyzed and/or oxidized.

In certain embodiments, the procedures comprise parboiling (blanching). After washing, certain plant parts may undergo a parboiling or blanching process in which they are put into boiling water for a brief period without being fully cooked. Such a heating procedure may serve several purposes, such as improving storage life of the processed materials by gelatinizing the starch, preventing mold or insect contamination, easily drying, destroying enzyme activity to prevent the alteration of certain chemical constituents, and facilitating further processing such as removal of the seed coat of almonds.

In certain embodiments, the procedures comprise boiling or steaming. The boiling process involves cooking the preparations in water or another liquid such as vinegar, wine, milk or other vehicle. In the steaming process, preparations are kept separate from the boiling water but have direct contact with the steam, resulting in a moist texture of the preparations. Often, the preparations are placed in a steamer or in a special utensil equipped with a flat frame suspended over boiling water. In certain embodiments, the preparations are pre-mixed with excipient substances such as wine, brine or vinegar before being steamed. The boiling or steaming process serves to soften plant tissues, to denature enzymes present in the preparations, and/or to thermally degrade selected chemical constituents. At the same time, the excipient, if used, is absorbed into the plant tissues to become an integral part of the processed preparations.

In certain embodiments, the procedures comprise baking or roasting. The baking or roasting process is a dry-heating using indirect, diffused heat, where the preparations are put in a heating device. The preparations are often embedded in bran or magnesium silicate (talc) powder to ensure even heating over the entire surface at an elevated temperature for a specified period of time. Some preparations are wrapped in moistened papers during the roasting process. The exact temperature used and duration of baking or roasting vary from one preparation to another. Some are baked or roasted until the surface color turns yellowish brown; some may be further heated until charred.

In certain embodiments, the procedures comprise stir-frying. Stir-frying is a process in which the preparations are put in a pot or frying pan, continuously stirred or tossed for a period of time under heating until the external color changes, charred or even carbonized. Depending on the plant species, the stir-frying process may require the addition of adjuvants such as wine, vinegar, honey, saline and ginger juice, which would be infused into the herbal matrix to become an integral part of the processed preparation. To ensure even heating over the surface of the preparations, sand, rice, bran, talc or clay can be admixed with the preparation during stir-frying.

In certain embodiments, the procedures comprise fumigation. Fumigation with sulfur dioxide has been employed in post-harvest handling of some plant parts for the purpose of preserving color, improving fresh-looking appearance, bleaching, preventing the growth of insects and inhibiting decay caused by molds. The process has been frequently applied to preparations of light and bright colors to avoid “browning.” Due to concerns about the undesirable residues, this process may be avoided as far as possible. When a real need is identified, treatment may be carried out at the earliest possible stage and exclusively by adequately trained and qualified personnel, according to the specific recommendations for use.

In certain embodiments, the procedures comprise irradiation. In certain embodiments, irradiation or ultraviolet light can be used to eliminate or reduce microbial load of the preparations. The use of these procedures has to comply with the national and/or regional regulations.

In certain embodiments, the procedures comprise advanced cutting, sectioning and comminution. When thoroughly dried, the preparations are processed by cutting and sectioning into convenient or specific sizes and shapes or forms for storage, direct use as decoction slices or pieces, and/or for further processing for the manufacture of herbal preparations or herbal dosage forms.

In certain embodiments, a plant part as described herein may be used in fresh form, for example, without the drying procedure. In certain embodiments, a preparation of a plant part as described herein as “dried” (for example, the preparation of the dried root and rhizome of Glycyrrhiza uralensis Fisch.) is produced from a fresh form of the plant part, for example, without the drying procedure. In certain embodiments, when producing a pharmaceutical composition from one or more preparations described herein, the weight of such fresh form of the plant part may be determined using known methods by a skilled artisan from the weight of the corresponding preparation of the dried plant part as described herein.

In certain embodiments, the preparation of the grassy stem of Ephedra sinica Stapf as described herein is generated from the grassy stem of Ephedra sinica Stapf using one or more procedures described above. In certain embodiments, the preparation of the grassy stem of Ephedra sinica Stapf as described herein is produced using procedures comprising washing and drying. In certain embodiments, the preparation of the grassy stem of Ephedra sinica Stapf is stored in a cool and dry condition.

In certain embodiments, the preparation of the seed (nut) of Semen Armeniacae Amarum described herein is generated from the seed (nut) of Semen Armeniacae Amarum using one or more procedures described above. In certain embodiments, the preparation of the seed (nut) of Semen Armeniacae Amarum as described herein is produced using procedures comprising washing and drying. In certain embodiments, the preparation of the grassy stem of Ephedra sinica Stapf is stored in a cool and dry condition.

In certain embodiments, the preparation of the dried root and rhizome of Glycyrrhiza uralensis Fisch. described herein is generated from the dried root and rhizome of Glycyrrhiza uralensis Fisch. using one or more procedures described above. In certain embodiments, the preparation of the dried root and rhizome of Glycyrrhiza uralensis Fisch. as described herein is produced using procedures comprising washing and drying. In certain embodiments, the preparation of the grassy stem of Ephedra sinica Stapf is stored in a cool and dry condition.

In certain embodiments, the preparation of the seed kernel of Coix lacryma-jobi L. var. mayuen (Roman.) Stapf described herein is generated from the seed kernel of Coix lacryma-jobi L. var. mayuen (Roman.) Stapf using one or more procedures described above. In certain embodiments, the preparation of the seed kernel of Coix lacryma-jobi L. var. mayuen (Roman.) Stapf as described herein is produced using procedures comprising washing and drying. In certain embodiments, the preparation of the grassy stem of Ephedra sinica Stapf is stored in a cool and dry condition.

In certain embodiments, the preparation of the dried sclerotia of Poria cocos (Schw.) Wolf described herein is generated from the dried sclerotia of Poria cocos (Schw.) Wolf using one or more procedures described above. In certain embodiments, the preparation of the dried sclerotia of Poria cocos (Schw.) Wolf as described herein is produced using procedures comprising washing and drying. In certain embodiments, the preparation of the grassy stem of Ephedra sinica Stapf is stored in a cool and dry condition.

In certain embodiments, the preparation of the dried root of Pueraria lobata Ohwi described herein is generated from the dried root of Pueraria lobata Ohwi using one or more procedures described above. In certain embodiments, the preparation of the dried root of Pueraria lobata Ohwi as described herein is produced using procedures comprising washing and drying. In certain embodiments, the preparation of the grassy stem of Ephedra sinica Stapf is stored in a cool and dry condition.

5.3.2 Method of Producing the Pharmaceutical Compositions from the Preparations

In certain embodiments, the pharmaceutical compositions are produced from one or more preparations of plant parts. Common procedures include but are not limited to extraction, distillation, fractionation, concentration, fermentation, or other physicochemical or biological methods. In certain embodiments, the resulting pharmaceutical compositions are in the forms of extracts, decoctions, tinctures, essential oils and others.

In certain embodiments, the pharmaceutical composition is produced using various procedures known in the art, for example, methods described in WHO guidelines on good herbal processing practices (GHPP) for herbal medicines (WHO Expert Committee on Specifications for Pharmaceutical Preparations, fifty-second report, WHO Technical Report Series, No. 1010 (2018)), Chinese Medicine Dictionary (2^{nd} edition, Editor, NAN JING ZHONG YI YAO DA XUE; Shanghai Science and Technology Press, China (1979)), and editions of Chinese Pharmacopoeia (ChP) (compiled by the Pharmacopoeia Commission of the Ministry of Health of the People's Republic of China, for example 10^{th} edition (2015)). Exemplary procedures include but are not limited to extraction, distillation, fractionation, concentration, fermentation, or other physicochemical or biological methods.

(i) Extraction

In certain embodiments, the pharmaceutical composition is produced from procedures comprising extraction. Extraction is a process in which soluble plant chemical constituents (including those which have therapeutic or prophylactic activity) are separated from insoluble plant metabolites and cellular matrix, by the use of selective solvent. The purpose of extraction of preparation is to eliminate unwanted materials and to concentrate other chemical constituents in a soluble form. Herbal extracts include liquid (fluid) extracts, soft extracts, oleoresins, dry extracts and others. The pharmaceutical composition so obtained may be ready for use as medicinal agents (for example in the form of decoction), or they may be further processed into herbal dosage forms such as tablets and capsules.

In certain embodiments, extraction is carried out using various techniques, including but are not limited to maceration, infusion, digestion, percolation (including hot continuous extraction) and decoction. Other extraction techniques can also be applied, for example, heat reflux extraction, counter-current extraction, microwave-assisted extraction, ultrasonic extraction (sonication) and supercritical fluid extraction. In certain embodiments, the pharmaceutical composition is produced using known extraction technology, extraction conditions, extraction solvents, ratio between preparation and solvents, and type of equipment in the art.

In certain embodiments, the following steps are involved prior to extraction: comminution, fragmentation, grinding or milling. In certain embodiments, the extraction process is carried out in the selected solvent at a desirable temperature for an optimal period of time. Depending on the polarity of the desired chemical constituents, water or other solvents can be used, either at room temperature (“cold” extraction) or at an elevated temperature (“hot” extraction).

In certain embodiments, sequential extraction with a series of solvents of differing polarity is sometimes done to create a series of extract fractions. In this procedure, preparation is subjected to organic and aqueous solvents in a sequence of increasing polarities, for example, n-hexane, dichloromethane, ethyl acetate, water-saturated n-butanol and water. As a result, chemical constituents possessing different polarities are transferred from the preparation to different solvent fractions according to the principle of “like dissolves like.” For example, the initial step of extraction using non-polar solvents (such as n-hexane or petroleum ethers) removes lipophilic constituents (such as alkanes, fatty acids and sterols) from the preparation in a process sometimes referred to as “defatting.” The compounds with intermediate polarity (such as flavonoid and quinone aglycones) will dissolve in the medium-polarity solvents (such as dichloromethane and ethyl acetate), whereas more polar compounds (such as glycosides and polyphenols) will be concentrated in the more polar solvents (such as butanol or water).

In certain embodiments, separation techniques are involved after extraction. The liquid so obtained is separated from the marc by filtration through a filter cloth or filter-paper to remove any particulate insoluble residues. Other separation techniques, including decantation, centrifugation or straining, may be used depending on the method of extraction and composition of the matrix.

In certain embodiments, the extract is concentrated by the removal of excessive solvent to a thick concentrated extract or to a solid mass. The concentration procedures may involve evaporation under reduced pressure, freeze-drying or spray-drying.

In certain embodiments, the extraction process using suitable solvents can yield herbal extracts of liquid, semi-solid or solid consistency. In certain embodiments, there are four general categories of herbal extracts, i.e. liquid (fluid) extract, soft extract, oleoresin and dry extract.

Liquid (fluid) extracts include but are not limited to fluidextract, decoction infusion, tincture, and macerate. In certain embodiments, fluidextract is an alcoholic liquid extract produced by percolation of preparation(s), for example, 1 mL of the fluidextract contains the extractive obtained from 1 g of the preparation(s). In certain embodiments, infusion is a dilute solution prepared by steeping the preparations in boiling water for a short time. In certain embodiments, tincture is an alcoholic or hydroalcoholic extract of a preparation, typically made up of 1 part preparation and 5 to 10 parts solvent (for example, ethanol or wine). In certain embodiments, macerate is a liquid preparation prepared by soaking the preparation(s), reduced to a suitable size, in water at room temperature for a defined period of time, usually for 30 minutes, when not otherwise specified.

In certain embodiments, the liquid extract is decoction. In certain embodiments, decoction is a water-based herbal preparation made by boiling preparations with water, and is commonly utilized in various traditional medicine contexts. In certain embodiments, aqueous ethanol or glycerol can also be used to prepare decoctions. In certain embodiments, decoctions may be prepared by a programmable decocting machine that processes the preparation at a specific temperature for a specific duration and then dispenses the decoction in hermetically sealed plastic pouches of a specified single-dosage volume that can be refrigerated for subsequent reheating and consumption.

In certain embodiments, the extraction process may yield soft extract, which is a semi-solid preparation obtained by total or partial evaporation of the solvent from a liquid extract. In certain embodiments, the extraction process may yield oleoresin, which is a semi-solid material composed of a resin in solution in an essential and/or fatty oil obtained by evaporation of the excess solvent. In certain embodiments, the extraction process may yield dry extract, which is a solid preparation obtained by evaporation of the solvent from a liquid/fluid extract. In certain embodiments, dry extract can also be prepared by spray-drying with or without the use of an adsorbent (such as methyl cellulose), or by drying and milling to produce a powder. In certain embodiments, the resulting power is further processed by compression or with use of a binding agent or granulation liquid to produce granules.

(ii) Distillation

In certain embodiments, the pharmaceutical composition is produced from procedures comprising distillation. For the extraction of volatile components of preparations, such as essential (volatile) oils, the odorous and volatile principles of plants, techniques such as distillation, expression and enfleurage may be employed. In certain embodiments, primary distillation takes place soon after the herb is harvested or collected to obtain crude oils. In other embodiments, preparations are distilled under well-controlled GMP conditions in the manufacturing facility. In certain embodiments, water or steam distillation is a method of choice for extracting volatile ingredients from herbs. In certain embodiments, the preparation is packed in a still, a sufficient amount of water is added and brought to the boil (water distillation). In certain embodiments, a stream of steam is introduced to the preparation that has been pre-soaked in water (water-steam distillation), or a stream of steam is introduced to preparations without water being added (direct steam distillation).

The method of distillation depends on the condition of the preparations. Water distillation can be applied to fresh herbs to avoid steam penetrating into the materials such as rose flowers, while direct steam distillation is often used for fresh or dried preparations. Freed from the plant tissue, the essential oil is carried away with the steam. Upon condensation, the water and oil are collected in liquid form, which then separates into two immiscible layers. During the process, the yield of essential oil can be quantified by using appropriate methods such as the Clevenger apparatus.

(iii) Fractionation

In certain embodiments, the pharmaceutical composition is produced from procedures comprising fractionation. Fractionation is a separation process in which a mixture is divided into a number of smaller quantities (fractions) with higher content of target substances (chemical compounds). The crude extracts of preparations contain complex mixtures of chemical constituents with diverse chemical and physical characteristics. It is often desirable to divide the chemical constituents into different groups based on their similarities in terms of chemical and physical properties, such as a flavonoid- or alkaloid-rich fraction. Fractional separation of a herbal extract can be achieved by subjecting the extract to a variety of fractionation techniques such as liquid-liquid partition and various forms of chromatography. The method can be applied to produce preparations enriched in active compounds, or to remove inactive and/or toxic constituents.

(iv) Concentration and Drying

In certain embodiments, the pharmaceutical composition is produced from procedures comprising concentration and drying.

In certain embodiments, the herbal extracts or fractions enriched in active ingredients are often reduced to produce a more concentrated liquid by the removal of excess solvent. This can be achieved through evaporation or vaporization. Solvent (single) can be recovered and may be reused provided that appropriate quality control is ensured. In certain embodiments, the concentration depends on the desired end product. Equipment for concentration may include descending film, thin layer or plate concentrators. Any method used to concentrate the extracts must avoid excessive heat because the active ingredients may be heat labile. The liquid preparation so obtained may be used as it is or further processed into a semisolid or dry extract.

When complete drying is required, the drying process can make use of vacuum freeze-dryers (lyophilizers), cabinet vacuum dryers, continuously operating drum or belt dryers, microwave ovens or atomizers. The choice of technique for drying depends on the stability of the product and the amount of solvent that must be removed. The total removal of solvent results in a dry extract, which may be less susceptible to microbial contamination than liquid extracts. Dry extract powders are often produced by drying the extract onto an inert carrier, such as methyl cellulose, maltodextrin or another excipient fit for the intended purpose, to facilitate processing into the final finished product.

(v) Fermentation

In certain embodiments, the pharmaceutical composition is produced from procedures comprising fermentation. In certain embodiments, the pharmaceutical composition is obtained after undergoing a process of fermentation of the comminuted preparation or decoction. Fermentation can be either natural (“self-fermentation”) involving microbial cultures already present on the herb, enzymes naturally occurring in the herb (which may be activated by bruising the herb), or both, or by introducing an appropriate microbial organism (for example, Lactobacillus bacteria or yeast). For natural fermentation, the dry comminuted preparation, a decoction, or an extract of preparation is often mixed with the juice of sugarcane, brown sugar or honey and the mixture is kept in an airtight utensil for several weeks for anaerobic fermentation to occur. In certain embodiments, preparations are mixed with a small amount of water and shaped into bricks, followed by microbial cultivation in an incubation room for a week or so, letting the mold grow on the surface of the preparations.

In certain embodiments, the pharmaceutical composition is produced in the form of decoction. In certain embodiments, the method comprises the steps of (a) boiling preparations as described in Section 5.1 in water; and (b) collecting the decoction. In certain embodiments, the method further comprises, before step (a), a step of soaking preparation in water for a period of time. In certain embodiments, the method further comprises, after step (b), a step of mixing water with the remaining preparations, boiling the mixture of water and the remaining preparations, collecting the second decoction, and mixing the second decoction with the decoction collected in step (b).

In certain embodiments, provided herein is a method of producing a pharmaceutical composition, wherein the method comprises the steps of:

(a) boiling preparations in water; and
(b) collecting the decoction,
wherein the preparations comprise:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

In certain embodiments, the pharmaceutical composition is produced in the form of granule. In certain embodiments, the method comprises the steps of (a) boiling one or more preparations as described in Section 5.1 in water; (b) collecting the decoction; (c) filtering the decoction and collecting the filtrate; and (d) freeze-drying the filtrate to obtain a dry power. In certain embodiments, the method further comprises, after step (b) and before step (c), a step of boiling the remaining preparations in water again, collecting the second decoction, and mixing the second decoction with the decoction collected in step (b) to produce the decoction in step (c). In certain embodiments, after each step of boiling and before each step of collecting the decoction, the preparations in water are maintained at a temperature of about 60° C. to about 100° C. for a period of about 30 minutes to about 120 minutes. In certain embodiments, the method further comprises, after step (c) and before step (d), a step of concentrating the filtrate under reduced pressure. In certain embodiments, the method further comprises, after step (d), a step of mixing the dry power with an encapsulating agent to produce a granule. In certain embodiments, the encapsulating agent is beta-cyclodextrin.

In certain embodiments, provided herein is a method of producing a pharmaceutical composition, wherein the method comprises the steps of:

(a) boiling preparations in water;
(b) collecting the decoction;
(c) filtering the decoction and collecting the filtrate; and
(d) freeze-drying the filtrate to obtain a dry powder;
wherein the preparations comprise:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

### 5.4 Assays

A person having ordinary skill in the art may use the assays as described herein and/or techniques known in the art to study the compositions and methods described herein, for example to evaluate the pharmaceutical composition as described in Section 5.1. The examples provided in Section 6 also demonstrate in more detail how such assays may be used.

### 5.4.1 Cell-Based Assays

(i) Antiviral Assay

The antiviral (for example, anti-influenza virus or anti-coronavirus) activity of a pharmaceutical compositions is examined using cytopathogenic effect (CPE) and MTT assays. Briefly, lung bronchial cells, for example, BEAS-2B, A549, or Madin-Darby Canine Kidney (MDCK) cells, are infected with viruses, for example, influenza virus or coronavirus (e.g., SARS-CoV-2), and treated with a pharmaceutical composition described herein in various concentrations. Cytotoxicity is measured using cytopathogenic effect (CPE) and MTT assays.

(ii) Plaque Reduction Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After incubation, the cell monolayer is covered with overlay medium containing a pharmaceutical composition described herein and further cultured. Subsequently, the overlay medium is removed, and the cell monolayer is fixed with 10% formalin, stained with 1% crystal violet, and the plaques are counted.

(iii) Time Course Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After infection, the supernatant is removed and the cells are rinsed. Then, a pharmaceutical composition described herein is added to cells at 0, 2, 4, 6, 8 and 10 hours after infection. The time-of-addition studies are conducted under single-cycle virus growth conditions. The supernatant is harvested at 12 hours post infection, and the virus titers are determined.

(iv) Indirect Immunofluorescence Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After infection, the supernatant is removed and the cells are rinsed. A pharmaceutical composition described herein is added to the cells. At 6 and 8 hours post infection, the cells are fixed and blocked, then incubated with antibody (for example, anti-influenza A virus NP antibody or anti-SARS-CoV-2 antibody) overnight at 4° C. After further washing, the cells are incubated with FITC-labeled goat anti-mouse IgG at 37° C. for 1 hour. The nuclei are stained with DAPI (5 μg/mL), and fluorescence is visualized using a Zeiss Axiovert 135 fluorescence microscope.

5.4.2 In Vivo Assays
(i) Mouse Inoculation and Anti-Viral Treatment

The mice are intranasally infected with viruses, for example, influenza virus or coronavirus (e.g., SARS-CoV-2). Groups of mice are orally administered varied dose of a pharmaceutical composition described herein daily. For example, a pharmaceutical composition is administered twice a day (at 12-hour intervals) for 5-7 days. The control animals are treated with the solvent only or an existing therapy.

(ii) Sample Collection, Process and Detection

One set of mice is monitored for weight loss from 3 days before to 15 days post infection of the virus. Any symptom or altered behavior of mice is recorded. A second set of animals is sacrificed at 4, 6, and 8 days after infection and the lung samples are harvested. Lung tissues from euthanized mice are extracted and homogenized. The lung homogenates are determined according to the virus titer using end-point titration in in vitro cell culture models and real-time RT-PCR for mRNA expression.

(iii) Histopathological Analysis

The lungs are inflated with 10% formaldehyde solution. The tissues are processed for paraffin embedding and cut into 4-μm-thick sections. The tissue samples are subjected to standard hematoxylin and eosin staining.

5.4.3 Clinical Studies

Participants take a pharmaceutical composition orally, for example, twice a day (e.g., in the morning and at night).

To study a pharmaceutical composition in the prevention of a respiratory disease and/or a viral infection as described in Section 5.2. Subjects can be a subject who has never been diagnosed with the respiratory disease and/or the viral infection; a subject who has been diagnosed with the respiratory disease and/or the viral infection previously and has recovered; and/or a subject who has been exposed to the viral infection (for example, a health care worker). The following outcomes can be evaluated: incidence of the disease, symptoms or lack thereof (for example, as recorded in a daily diary), and/or proportion of participants with no evidence of disease in comparison to no treatment.

To study a pharmaceutical composition in the treatment and/or management of a respiratory disease and/or a viral infection as described in Section 5.2. The following outcomes can be evaluated: nucleic-acid based viral test, number of subject being hospitalized due to the diseases, improvement in symptoms due to the diseases (for example, body temperature, shortness of breath, cough, headache, and muscle pain), number of subject having severe symptoms due to the diseases, and/or number of subject having respiratory failure due to the diseases. Other outcomes can be: a change from baseline (for example, pre-dose) viral load (for example, SAR-CoV-2 load) from patient samples (for example, nasopharyngeal samples) on certain days post starting of treatment (for example, 3, 4, 5, 6, 7, 8, 9, 10, 11, 12, 13, and 14 days post starting of treatment) and a difference between the treatment group and the control group (for example, placebo group) in the viral load AUC (Area under the Curve) during certain days (for example, days 1-3; days 1-4, days 1-5, days 1-6, days 1-7, days 1-8, days 1-9, days 1-10, days 1-11, days 1-12, days 1-13, and days 1-14).

Another outcome is the odds of ratio for improvement on a 7-point ordinal Scale on certain day post starting treatment, for example 14 days. The odds ratio represents the odds of improvement in the ordinal scale between the treatment groups. The ordinal scale is an assessment of the clinical status at a given day. Each day, the worst score from the previous day will be recorded. The scale is as follows: 1. Death; 2. Hospitalized, on invasive mechanical ventilation or Extracorporeal Membrane Oxygenation (ECMO); 3. Hospitalized, on non-invasive ventilation or high flow oxygen devices; 4. Hospitalized, requiring low flow supplemental oxygen; 5. Hospitalized, not requiring supplemental oxygen—requiring ongoing medical care (disease related or otherwise); 6. Hospitalized, not requiring supplemental oxygen—no longer required ongoing medical care besides the methods of treatment described herein; 7. Not hospitalized.

### 6. EXAMPLES

### 6.1 Example 1: Production of a Pharmaceutical Composition in the Form of Decoction

This example provides exemplary methods of producing a pharmaceutical composition provided herein in the form of decoction.

The preparations were weighted as the following: (i) the preparation of the grassy stem of Ephedra sinica Stapf in an amount of 10 g; (ii) Gypsum ore (CaSO₄.2H₂O) in an amount of 30 g; (iii) the preparation of the seed (nut) of Semen Armeniacae Amarum in an amount of 10 g; (iv) the preparation of the dried root and rhizome of Glycyrrhiza uralensis Fisch. in an amount of 10 g; (v) the preparation of the seed kernel of Coix lacryma-jobi L. var. mayuen (Roman.) Stapf in an amount of 15 g; (vi) the preparation of the dried sclerotia of Poria cocos (Schw.) Wolf in an amount of 20 g; and (vii) the preparation of the dried root of Pueraria lobata Ohwi in an amount of 20 g.

The preparations were placed into a pot, into which cold water was added. The preparations were soaked in the water for about 20 minutes, and then boiled in water covering the surface of the preparations. The preparations were boiled once or twice. The first boiling took about 20 to 30 minutes, then the decoction in the pot was collected. The second boiling took about 8 to 10 minutes. The decoction in the pot were collected and mixed with the decoction collected from the first boiling.

### 6.2 Example 2: Production of a Pharmaceutical Composition in the Form of Granule

This example provides exemplary methods of producing a pharmaceutical composition provided herein in the form of granule.

The preparations were weighted as the following: (i) the preparation of the grassy stem of Ephedra sinica Stapf in an amount of 10 g; (ii) Gypsum ore (CaSO₄.2H₂O) in an amount of 30 g; (iii) the preparation of the seed (nut) of Semen Armeniacae Amarum in an amount of 10 g; (iv) the preparation of the dried root and rhizome of Glycyrrhiza uralensis Fisch. in an amount of 10 g; (v) the preparation of the seed kernel of Coix lacryma-jobi L. var. mayuen (Roman.) Stapf in an amount of 15 g; (vi) the preparation of the dried sclerotia of Poria cocos (Schw.) Wolf in an amount of 20 g; and (vii) the preparation of the dried root of Pueraria lobata Ohwi in an amount of 20 g.

The preparations were place in a closed container and boiled twice. During the first boiling process, water was added in an amount that was 4-12 times (commonly 8 times) of the total weight of the pharmaceutical composition of the preparations. After boiling, the mixture was maintained at 80° C. for 60 minutes, following which the decoction was obtained. During the second boiling process, water was added in an amount that was 4-10 times (commonly 6 times) of the total weight of the pharmaceutical composition of the preparations. After boiling, the mixture was maintained at 80° C. for 60 minutes, following which the decoction was obtained and combined with the decoction collected from the first boiling. The combined decoction was filtered and the filtrate was collected and concentrated under reduced pressure (−0.05 MPa, 60° C.) to a relative density of 1.05-1.15 (60° C.). The concentrated filtrate was freeze-dried under reduced pressure to obtain a dry extract powder, and the extraction rate is 25% of the crude preparations.

An appropriate amount of auxiliary materials, for example, β-cyclodextrin, or other pharmaceutically acceptable auxiliary materials, was mixed with the dry extract power at a ratio of 4:1 to produce dry extract powder particles. The granules were dried and cooled. The granules were packed in bag, for example, in the amount of 1250 mg of dry extract powder (equivalent to 20 g of the crude preparations) per bag. Alternatively, the granules were produced into capsules or tablets. Each capsule or tablet contained 312.5 mg of the dry extract powder (equivalent to 5 g of the crude preparations).

The granules can be produced into forms including but not limited to: soup, tablet, sugar-coated tablet, film-coated tablet, enteric-coated tablet, capsule, hard capsule, soft capsule, oral liquid, granule, pill, powder, ointment, suspension, solution, injection, suppository, plaster, cream, spray, drop, and patch agent.

### 6.3 Example 3: Treatment and Management of COVID-19 Using the Pharmaceutical Compositions

This example provides an exemplary method of treating and/or managing COVID-19 using the pharmaceutical compositions produced herein.

A total of 9 subjects participated in the study. Participants were provided the pharmaceutical compositions produced in Examples 1 and 2 and instructed to take the pharmaceutical compositions orally for 7 days. For example, an participant could take the pharmaceutical composition as produced in Example 1 in the same amount twice a day (e.g., in the morning and at night). An participate could take the granules produced in Example 2 twice a day and 2-4 bags each time.

TABLE 1
Outcomes of Treatment

| | Number of Subjects | |
|---|---|---|
| Characteristic | Before Treatment | After Treatment |
| COVID-19 Nucleic-acid based Test | | |
| Positive | 9 | 3 |
| Negative | 0 | 6 |
| Body temperature | | |
| >100.5° F. | 9 | 0 |
| 98.6-100.5° F. | 0 | 2 |
| <98.6° F. | 0 | 7 |
| Shortness of breath | | |
| Yes | 9 | 1 |
| No | 0 | 8 |
| Cough | | |
| Yes | 9 | 1 |
| No | 0 | 8 |
| Headache | | |
| Yes | 7 | 2 |
| No | 2 | 7 |
| Muscle Pain | | |
| Yes | 7 | 1 |
| No | 2 | 8 |

### 6.4 Example 4: Cell-Based Assays

This prophetic example provides exemplary methods of cell-based assays that could be used to study the pharmaceutical compositions provided herein.

6.4.1 Antiviral Assay

The antiviral (for example, anti-influenza virus or anti-coronavirus) activity of a pharmaceutical composition described herein is examined using cytopathogenic effect (CPE) and MTT assays. Briefly, lung bronchial cells, for example, BEAS-2B, A549, or Madin-Darby Canine Kidney (MDCK) cells, are infected with viruses, for example, influenza virus or coronavirus (e.g., SARS-CoV-2), and treated with a pharmaceutical composition described herein in various concentrations. Cytotoxicity is measured using cytopathogenic effect (CPE) and MTT assays.

6.4.2 Plaque Reduction Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After incubation, the cell monolayer is covered with overlay medium containing a pharmaceutical composition described herein and further cultured. Subsequently, the overlay medium is removed, and the cell monolayer is fixed with 10% formalin, stained with 1% crystal violet, and the plaques are counted.

6.4.3 Time Course Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After infection, the supernatant is removed and the cells are rinsed. Then, a pharmaceutical composition described herein is added to cells at 0, 2, 4, 6, 8 and 10 hours after infection. The time-of-addition studies are conducted under single-cycle virus growth conditions. The supernatant is harvested at 12 hours post infection, and the virus titers are determined.

6.4.4 Indirect Immunofluorescence Assay

Lung bronchial cells, for example, BEAS-2B, A549, or Madin-Darby Canine Kidney (MDCK) cells, are cultured and infected with viruses, for example, influenza virus or coronavirus (e.g., SARS-CoV-2). After infection, the supernatant is removed and the cells are rinsed. A pharmaceutical composition described herein is added to the cells. At 6 and 8 hours post infection, the cells are fixed and blocked, then incubated with antibody (for example, anti-influenza A virus NP antibody or anti-SARS-CoV-2 antibody) overnight at 4° C. After further washing, the cells are incubated with FITC-labeled goat anti-mouse IgG at 37° C. for 1 hour. The nuclei are stained with DAPI (5 μg/mL), and fluorescence is visualized using a Zeiss Axiovert 135 fluorescence microscope.

### 6.5 Example 5: In Vivo Assays

This prophetic example provides exemplary methods of in vivo assays that could be used to study the pharmaceutical compositions provided herein.

6.5.1 Mouse Inoculation and Anti-Viral Treatment

The mice are intranasally infected with viruses, for example, influenza virus or coronavirus (e.g., SARS-CoV-2). Groups of mice are orally administered varied dose of a pharmaceutical composition described herein daily. For example, a pharmaceutical composition is administered twice a day (at 12-hour intervals) for 5-7 days. The control animals are treated with the solvent only or an existing therapy.

6.5.2 Sample Collection, Process and Detection

One set of mice is monitored for weight loss from 3 days before to 15 days post infection of the virus. Any symptom or altered behavior of mice is recorded. A second set of animals is sacrificed at 4, 6, and 8 days after infection and the lung samples are harvested. Lung tissues from euthanized mice are extracted and homogenized. The lung homogenates are determined according to the virus titer using end-point titration in in vitro cell culture models and real-time RT-PCR for mRNA expression.

6.5.3 Histopathological Analysis

The lungs are inflated with 10% formaldehyde solution. The tissues are processed for paraffin embedding and cut into 4-μm-thick sections. The tissue samples are subjected to standard hematoxylin and eosin staining.

### 7. EQUIVALENTS

The pharmaceutical compositions and methods disclosed herein are not to be limited in scope by the specific embodiments described herein. Indeed, various modifications of methods and pharmaceutical compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method of preventing, treating or managing a respiratory disease and/or a viral infection, comprising administering to a subject in need of such prevention, treatment, or management a pharmaceutical composition produced from one or more preparations comprising
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

2. The method according to claim 1, wherein the respiratory disease and/or the viral infection is pneumonia, an influenza virus infection, or a coronavirus infection.

3.-6. (canceled)

7. The method according to claim 2, wherein the coronavirus infection is coronavirus disease 2019 (COVID-19).

8.-12. (canceled)

13. The method according to claim 1, wherein the method further comprises administering to the subject a second agent.

14. The method according to claim 13, wherein the second agent is an antiviral agent, an anti-inflammatory agent, a Janus kinase inhibitor, a steroid hormone, an immunosuppressive agent, an agent targeting interleukin-6 receptor, an antibacterial agent, an anti-parasitic agent, Traditional Chinese medicine, stem cells, convalescent plasma, or a combination thereof.

15. (canceled)

16. The method according to claim 1, wherein the pharmaceutical composition is administered via oral route.

17. The method according to claim 1, wherein the preparations comprise:
(i) the preparation of the grassy stem of Ephedra sinica Stapf in an amount of 1 wt. % to 50 wt. % or 2 g to 20 g;
(ii) Gypsum ore (CaSO₄.2H₂O) in an amount of 5 wt. % to 80 wt. % or 15 g to 60 g;
(iii) the preparation of the seed (nut) of Semen Armeniacae Amarum in an amount of 2 wt. % to 50 wt. % or 5 g to 20 g; and/or
(iv) the preparation of the dried root and rhizome of Glycyrrhiza uralensis Fisch. in an amount of 1 wt. % to 50 wt. % or 3 g to 20 g.

18.-19. (canceled)

20. The method according to claim 1, wherein the preparations further comprise:
(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

21. The method according to claim 1, wherein the preparations comprise:
(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. %;
(vi) the preparation of the dried sclerotia of Poria cocos (Schw.) Wolf in an amount of 1 wt. % to 60 wt. %; and
(vii) the preparation of the dried root of Pueraria lobata Ohwi in an amount of 1 wt. % to 60 wt. %.

22.-23. (canceled)

24. A pharmaceutical composition produced from preparations comprising:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

25. The pharmaceutical composition according to claim 24, wherein the preparations comprise:
(i) the preparation of the grassy stem of Ephedra sinica Stapf in an amount of 1 wt. % to 50 wt. % or 2 g to 20 g;
(ii) the preparation of the Gypsum ore (CaSO₄.2H₂O) in an amount of 5 wt. % to 80 wt. % or 15 g to 60 g;
(iii) the preparation of the seed (nut) of Semen Armeniacae Amarum in an amount of 2 wt. % to 50 wt. % or 5 g to 20 g; and/or
(iv) the preparation of the dried root and rhizome of Glycyrrhiza uralensis Fisch. in an amount of 1 wt. % to 50 wt. % or 3 g to 20 g.

26.-31. (canceled)

32. A method of producing a pharmaceutical composition, wherein the method comprises the steps of:
(a) boiling preparations in water; and
(b) collecting the decoction,
wherein the preparations comprise:
(i) a preparation of the grassy stem of Ephedra sinica Stapf;
(ii) Gypsum ore (CaSO₄.2H₂O);
(iii) a preparation of the seed (nut) of Semen Armeniacae Amarum; and/or
(iv) a preparation of the dried root and rhizome of Glycyrrhiza uralensis Fisch., and
wherein the pharmaceutical composition comprises a pharmaceutically acceptable carrier.

33.-40. (canceled)

41. The method according to claim 32, wherein the preparations comprise:
(i) the preparation of the grassy stem of Ephedra sinica Stapf in an amount of 1 wt. % to 50 wt. % or 2 g to 20 g;
(ii) Gypsum ore (CaSO₄.2H₂O) in an amount of 5 wt. % to 80 wt. % or 15 g to 60 g;
(iii) the preparation of the seed (nut) of Semen Armeniacae Amarum in an amount of 2 wt. % to 50 wt. % or 5 g to 20 g; and/or
(iv) the preparation of the dried root and rhizome of Glycyrrhiza uralensis Fisch. in an amount of 1 wt. % to 50 wt. % or 3 g to 20 g.

42.-43. (canceled)

44. The method according to claim 32, wherein the preparations further comprise:
(v) a preparation of the seed kernel of Coix lacryma-jobi L. var. mayuen (Roman.) Stapf, and
(vi) a preparation of the dried sclerotia of Poria cocos (Schw.) Wolf; and
(vii) a preparation of the dried root of Pueraria lobata Ohwi.

45. The method according to claim 44, wherein the preparations comprise
(i) the preparation of the grassy stem of Ephedra sinica Stapf in an amount of 1 wt. % to 50 wt. %;
(ii) Gypsum ore (CaSO₄.2H₂O) in an amount of 5 wt. % to 80 wt. %;
(iii) the preparation of the seed (nut) of Semen Armeniacae Amarum in an amount of 2 wt. % to 50 wt. %;
(iv) the preparation of the dried root and rhizome of Glycyrrhiza uralensis Fisch. in an amount of 1 wt. % to 50 wt. %;
(v) the preparation of the seed kernel of Coix lacryma-jobi L. var. mayuen (Roman.) Stapf in an amount of 4 wt. % to 40 wt. % or 9 g to 30 g;
(vi) the preparation of the dried sclerotia of Poria cocos (Schw.) Wolf in an amount of 1 wt. % to 60 wt. % or 5 g to 35 g; and
(vii) the preparation of the dried root of Pueraria lobata Ohwi in an amount of 1 wt. % to 60 wt. % or 5 g to 35 g.

46.-47. (canceled)

48. A pharmaceutical composition obtainable by the method according to claim 32.

49. A method of preventing, treating or managing a respiratory disease and/or a viral infection, comprising administering to a subject in need of such prevention, treatment, or management a pharmaceutical composition according to claim 48.

50. (canceled)
